(19) United States
(12) Patent Application Publication
(10) Pub. No.: US 2024/0189276 A1
(43) Pub. Date: Jun. 13, 2024

(54) COMPOSITIONS COMPRISING LACTATE DEHYDROGENASE-A INHIBITORS AND METHODS OF PRODUCTION AND USE THEREOF

(71) Applicant: Southwestern Oklahoma State University, Weatherford, OK (US)

(72) Inventors: Horrick Sharma, Weatherford, OK (US); Pragya Sharma, Weatherford, OK (US)

(21) Appl. No.: 18/060,323

(22) Filed: Nov. 30, 2022

(51) Int. Cl. A61K 31/4025; A61K 31/4155; A61P 35/00

(52) U.S. Cl. CPC A61K 31/4025; A61K 31/4155; A61P 35/00

(57) ABSTRACT

Compounds that incorporate a succinic acid scaffold are disclosed that inhibit Lactate dehydrogenase-A (LDHA). Also disclosed are pharmaceutical compositions containing these compounds. In addition, methods of using these compounds in methods of inhibiting LDHA expression and/or enzymatic activity in cells and methods of reducing viability of cancer cells are disclosed. Also disclosed are methods of treating a subject in need thereof with the pharmaceutical compositions containing one or more LDHA-inhibiting compounds.

### STATEMENT REGARDING FEDERALLY SPONSORED RESEARCH OR DEVELOPMENT

This invention was made with Government support under Grant No. P20GM103447 awarded by the National Institutes of Health. The Government has certain rights in the invention.

### CROSS REFERENCE TO RELATED APPLICATIONS/INCORPORATION BY REFERENCE STATEMENT

Not Applicable.

### BACKGROUND

Metabolic reprogramming is considered one of the hallmarks of cancer. Oncogenic signaling upregulates metabolic enzymes and nutrient transporters to sustain tumor cell growth and proliferation. Most tumor cells undergo a switch from oxidative phosphorylation (OXPHOS) to aerobic glycolysis (Warburg effect). Lactate dehydrogenase-A (LDHA) is a key glycolytic enzyme and a mediator of the Warburg effect. LDHA catalyzes the conversion of pyruvate to lactate and simultaneously reduces NADH to NAD+, which is required to drive glycolysis and ATP production in cancer cells. LDHA overexpression is shown to promote the progression of multiple tumors. LDHA-associated lactate production promotes angiogenesis, metastasis, and immunosuppression of tumor cells. In addition, lactate inhibits the activation of immune cells, including monocytes and T lymphocytes, and assists tumor cells in immune escape.

It has been suggested that LDHA inhibitors may improve the efficacy of immune checkpoint inhibitors. Compelling evidence in the last decade has established LDHA as a promising drug target that can also overcome resistance to current cancer chemotherapy and improve the efficacy of immunotherapy. LDHA is considered a safe drug target, as its genetic deficiency does not result in any adverse effect, except myoglobinuria after extreme exercise.

Recent efforts have led to the discovery of LDHA inhibitors, and certain examples of these identified LDHA inhibitors are shown in FIG. 1. However, these known LDHA inhibitors exhibit various limitations, such as (but not limited to) their lack of cellular activity or high clearance when tested in vivo. For example, the Astra Zeneca compound AZ33 exhibited a 50% inhibitory concentration (IC₅₀) of 0.5 μM for LDHA but was inactive in cells (Ward et al. (2012) J Med Chem, 55(7):3285-3306). Glaxo compound GSK2837808A displayed potent inhibition of LDHA (IC₅₀ 1.9 nM) and showed cellular potency (cell viability EC₅₀ of 2.9 μM in Snu398 cells); however, GSK2837808A suffered from poor pharmacokinetics in vivo (Billiard et al. (2013) Cancer & Metabolism, 1(1):1-19). Maloney et al. reported pyrazole derivatives (such as compound 63 in FIG. 1) as inhibitors of both LDHA and LDHB in the nano-molar range. However, the best compound of the series decreased viability of pancreatic MIAPaCa-2 cells with an IC₅₀ of 3.9 μM in a cytotoxicity assay (Rai et al. (2017) J Med Chem, 60(22):9184-9204). Genentech's GNE-140 also inhibited both LDHA and LDHB with IC₅₀s of 3 nM and 5 nM, respectively. However, despite much potent biochemical activity, GNE-140 inhibited cell viability in MIA PaCa-2 with IC₅₀ of 2.05 μM, probably due to poor penetration of the compound into the cell (Boudreau et al. (2016) Nat Chem Biol, 12(10):779-86). GNE-140 was tested to inhibit tumor growth in vivo, but this inhibitor suffered from high clearance due to its poor pharmacokinetic properties. The above limitations for the previously identified LDHA inhibitors are specific to their chemical structure, leading to low cellular penetration or greater plasma protein binding and should not be construed as a concern for developing novel LDH inhibitors. Thus, there is an urgent need to discover and develop novel chemical scaffolds that not only inhibit LDH in vitro but possess comparable activity in cells for inhibition of tumor growth and progression.

### BRIEF DESCRIPTION OF THE DRAWINGS

FIG. 1 illustrates the structures or potential Lactate Dehydrogenase-A (LDHA) Inhibitors currently known in the art.

FIG. 2 graphically illustrates a docked pose of one non-limiting embodiment of an LDHA inhibitor constructed in accordance with the present disclosure (LDI-7) in the LDHA active site.

FIG. 3 illustrates the structures of various non-limiting embodiments of LDHA inhibitors constructed in accordance with the present disclosure and designated as Formulas IX, X, XI, XII, and XIII (note that the structures of Formulas IX-XIII may also be referenced herein as LDI-7, LDI-4, LDI-5, LDI-17, and LDI-19, respectively).

FIG. 4 graphically illustrates the inhibition of LDHA and dose response curves for four of the LDHA inhibitors of FIG. 3.

FIG. 5 graphically illustrates a dose-response curve of compound LDI-7 (Formula IX) in a cytotoxicity assay in pancreatic cancer MIA PaCa-2, PNAC-1, and FC1199 cells. The graph represents a plot of dose (nM) vs. % untreated cells (Hoechst) after 48 hours of treatment.

FIG. 6 graphically illustrates dose-response curves for four non-limiting embodiments of LDHA inhibitors constructed in accordance with the present disclosure (designated herein as LDI-1, LDI-4, LDI-17, and LDI-23, which correspond to the structures of Formulas XLIV, X, LVII, and LXII, respectively).

FIG. 7 contains photomicrographs that illustrate morphological changes induced in MIA PaCa-2 cells upon treatment with the LDHA inhibitor LDI-7 (Formula IX) versus control after 48 hrs.

FIG. 8 contains a Western blot analysis of apoptotic proteins in MIS PaCa-2 cells treated with compound LDI-23 versus control.

FIG. 9 graphically illustrates the effects of compounds 23 and 4 on lactate production in MIA PaCa-2 cells following 6 hours of treatment under normoxic conditions in cell culture medium containing 25 mM Glucose, 2 mM glutamine, and 1 mM pyruvate.

FIG. 10 graphically depicts inhibition of lactate production by LDI-7 and LDI-22 (Formulas IX and XXXV, respectively) in MIA PaCa-2 cells upon 6 hours of treatment in (A) cell culture medium containing 25 mM Glucose, 2 mM glutamine, and 1 mM pyruvate; and (B) cell culture medium containing 10 mM Glucose, 2 mM glutamine, and without pyruvate. Lactate levels are reported as mean±SD. Data were statistically compared using one-way ANOVA. The experiment was repeated 3-4 times.

FIG. 11 illustrates the structures of a variety of non-limiting embodiments of LDHA inhibitors constructed in accordance with the present disclosure.

FIG. 12 illustrates a synthesis scheme for certain compounds of FIG. 11. Scheme 1. (a) MeOH, H2SO4, 80° C., 3 h; (b) BuLi, diisopropyl amine, THF, −78° C., tert-butyl bromoacetate 1 h; (c) TFA, DCM 4 h, rt; (d) Oxalyl chloride, DMF, DCM 0° C.-rt, 2 h, R2NH2 , Et3N, overnight; (e) LiOH, H2O, THF, rt, 3 h.

FIG. 13 illustrates the structures of a variety of additional non-limiting embodiments of LDHA inhibitors constructed in accordance with the present disclosure.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed herein can be made and executed without undue experimentation in light of the present disclosure. While the compositions and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The terms “analog,” “derivative,” or “variant” as used herein will be understood to refer to a variation of the normal or standard form or the wild-type form of molecules. For polypeptides, an analog may be a variant (polymorphism), a mutant, and/or a naturally or artificially chemically modified version of the wild-type polypeptide (including combinations of the above). Such analogs may have higher, full, intermediate, or lower activity than the normal form of the molecule, or no activity at all. Alternatively, and/or in addition thereto, for a chemical, an analog may be any structure that has the desired functionalities (including alterations or substitutions in the core moiety), even if comprised of different atoms or isomeric arrangements.

As used herein, “substantially pure” means an object species is the predominant species present (i.e., on a molar basis it is more abundant than any other individual species in the composition), and a substantially purified fraction is a composition wherein the object species comprises at least about 50 percent (on a molar basis) of all macromolecular species present. Generally, a substantially pure composition will comprise more than about 80 percent of all macromolecular species present in the composition, such as (but not limited to) more than about 85%, 90%, 95%, and 99%. In a particular (but non-limiting) embodiment, the object species is purified to essential homogeneity (contaminant species cannot be detected in the composition by conventional detection methods), wherein the composition consists essentially of a single macromolecular species.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “patient” or “subject” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent/element/method to a patient for therapeutic and/or prophylactic/preventative purposes.

The term “therapeutic composition” or “pharmaceutical composition” as used herein refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof, or an amount of a treatment protocol (e.g., an alternating electric field), sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of at least one condition, disease, and/or infection. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy,” and will be understood to mean that the patient in need of treatment is treated or given another drug for the condition/disease/infection in conjunction with the treatments of the present disclosure. This concurrent therapy can be sequential therapy, where the patient is treated first with one treatment protocol/pharmaceutical composition and then the other treatment protocol/pharmaceutical composition, or the two treatment protocols/pharmaceutical compositions are given simultaneously.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

Turning now to the inventive concept(s), certain non-limiting embodiments of the present disclosure are directed to a composition, comprising at least one compound, wherein the at least one compound is an inhibitor of Lactate dehydrogenase-A (LDHA). The LDHA-inhibitory compound may be provided with any structure disclosed in the application or drawings or otherwise contemplated herein; non-limiting examples thereof include the structures of Formulas I-LXXXIX.

In certain non-limiting embodiments, the at least one compound has a general structure represented by one or more of Formulas I, II, and III:

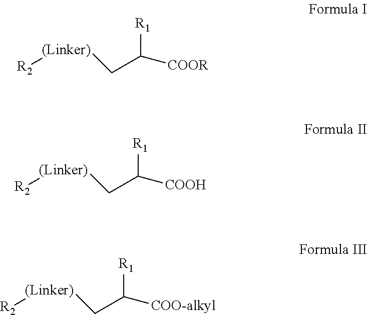

or an enantiomer, stereoisomer, or pharmaceutically acceptable salt thereof. In each of Formulas I-III, the R, R₁, R₂, and Linker groups are defined as follows. The R group is H, an alkyl group, or a substituted phenyl group. The R₁ group is selected from a phenyl group; an alkyl group; a substituted phenyl group; an aromatic ring; a heteroaromatic ring; a fused bicyclic group; a biphenyl group; or a 3-7 membered carbocyclic or alicyclic ring incorporating at least one heteroatom. In addition, the R₁ group is not a COOH group. The R₂ group is selected from a phenyl group; a substituted phenyl group; an aromatic ring; a heteroaromatic ring; a bicyclic group; a tricyclic group; a 3-7 membered alicyclic ring incorporating at least one heteroatom; a carbocyclic ring; or a disubstituted carbocyclic ring. The Linker group comprises at least one of an amide group; a reverse amide group; an ether group; an alkyl group; or a 3-7 membered carbocyclic, alicyclic, heterocyclic, aromatic, or heteroaromatic ring.

In certain particular (but non-limiting) embodiments, the at least one compound is further defined as having a general structure represented by one of Formulas IV, V, VI, or VII:

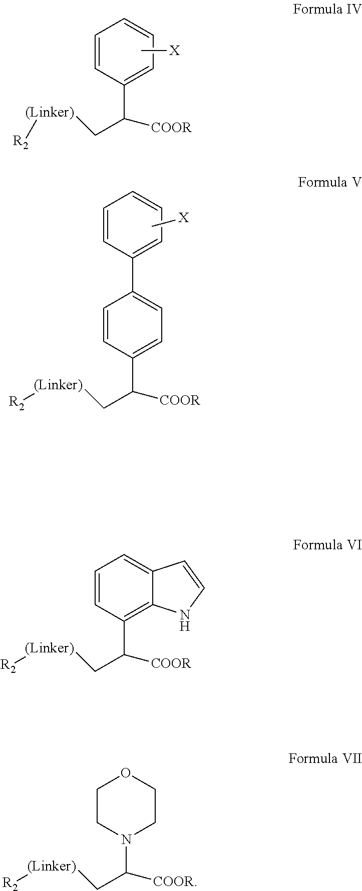

In the above formulas, X is selected from the group consisting of H and at least one ortho, meta, or para substitution. In certain particular (but non-limiting) embodiments, the at least one substitution of X is selected from the group consisting of a halogen, an alkyl group, a cyano group, a nitro group, a trifluoromethyl group, a hydroxyl group, an amino group, a carboxylic acid group, a carbocyclic ring, an alicyclic ring incorporating at least one heteroatom, a heteroaromatic ring, a substituted heteroaromatic ring, and combinations thereof.

In certain particular (but non-limiting) embodiments, the Linker of Formula(s) I, II, and/or III is an amide group. Non-limiting examples of structures of compounds of the present disclosure that have an amide linker include those having a general structure represented by Formula VIII or XXXI:

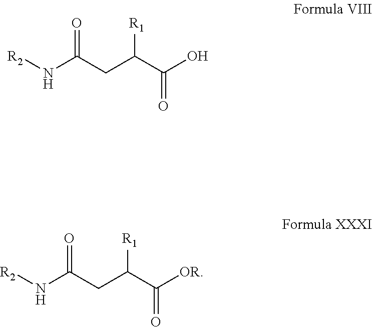

Particular (but non-limiting) examples of structures represented by Formula VIII or XXXI is Formula X, XII, LXII, and LXIII:

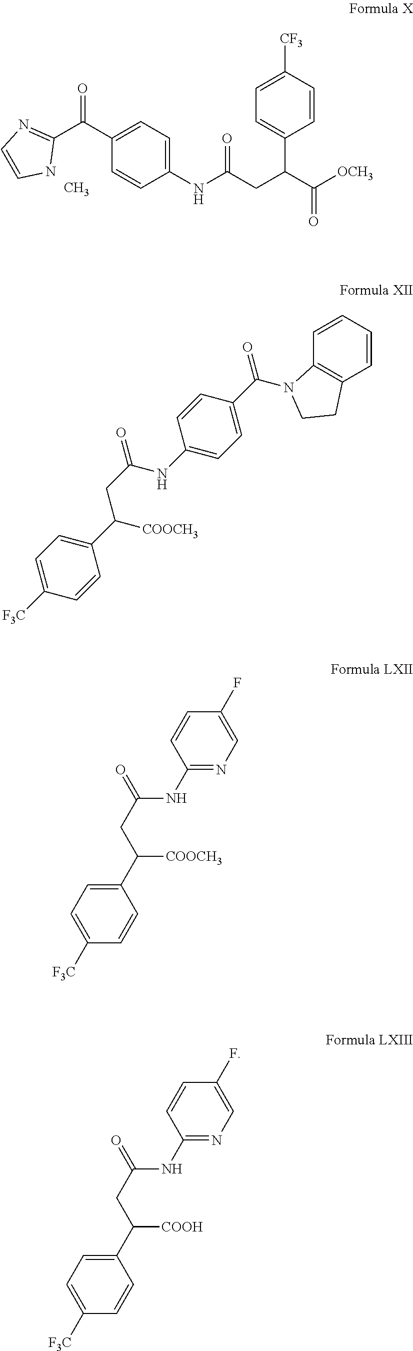

In certain particular (but non-limiting) embodiments, the Linker of Formula(s) I, II, and/or III comprises a heterocyclic ring as shown in Formula XIV or XX:

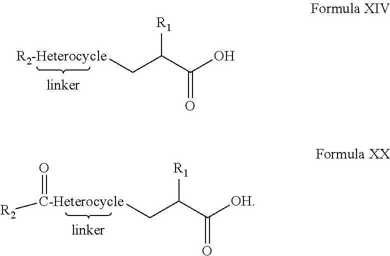

Non-limiting examples of structures of compounds of the present disclosure that have a heterocyclic ring linker include those having a general structure represented by Formula XV, XVI, XVII, XVIII, XIX, XXI, or XXII:

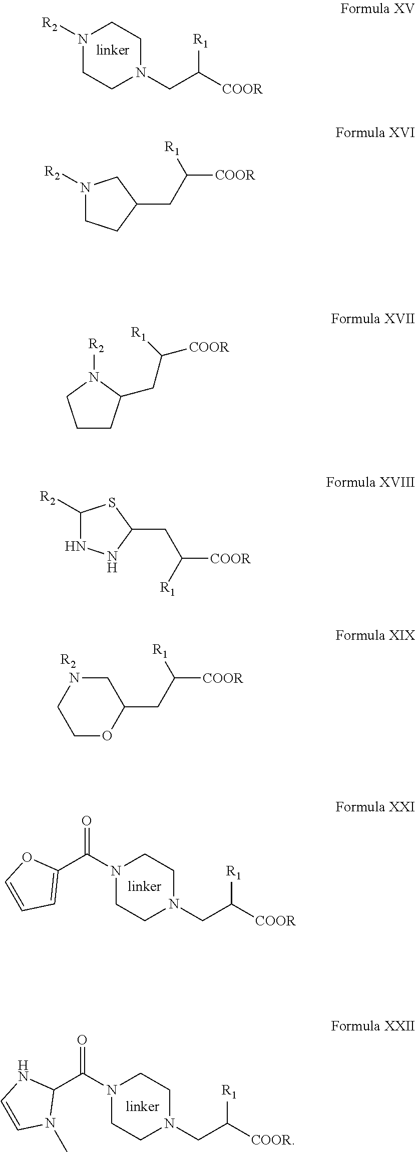

In certain particular (but non-limiting) embodiments, the Linker of Formula(s) I, II, and/or III comprises an amide group and a ring that is connected to a carbonyl group. Non-limiting examples of structures of compounds of the present disclosure that have such a linker include those having a general structure represented by Formula XXIII:

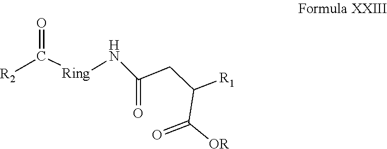

wherein the Ring comprises a 3-7 membered carbocyclic, alicyclic, heterocyclic, aromatic, or heteroaromatic ring. Particular (but non-limiting) examples of structures that meet Formula XXIII include structures represented by Formulas IX, XI, XIII, XLV, XXIV, XXV, XXVI, LIII, LIX, LX, LXXVI, LXXVIII, LXXXVIII, or LXXXIX:

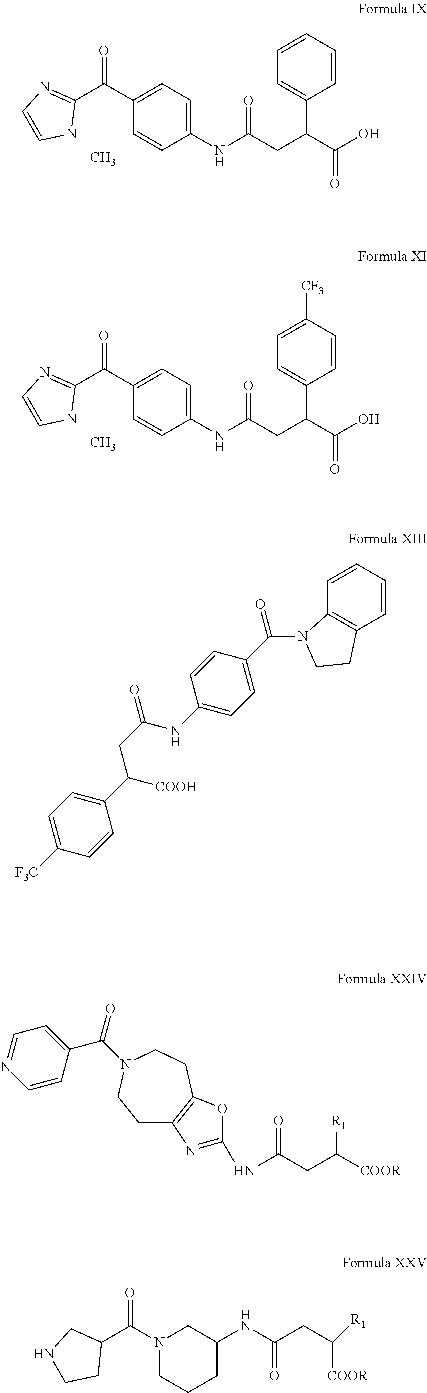

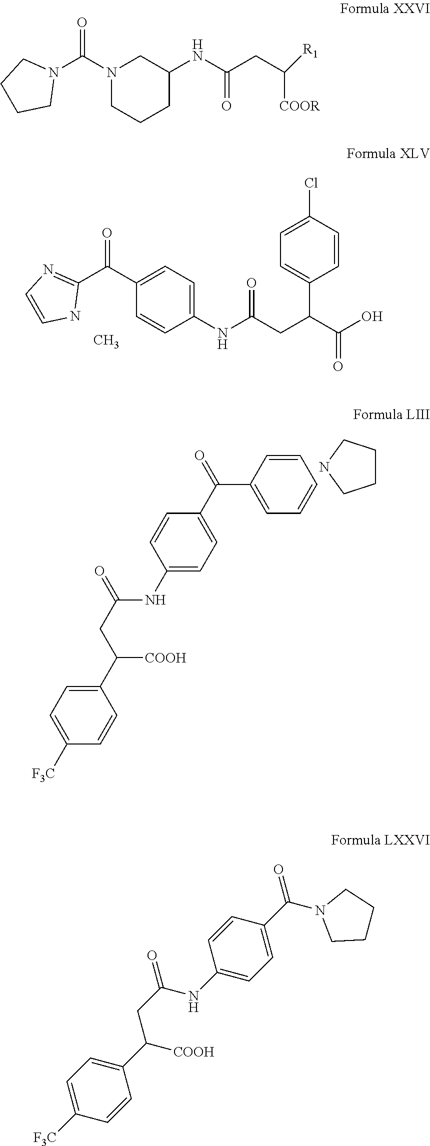

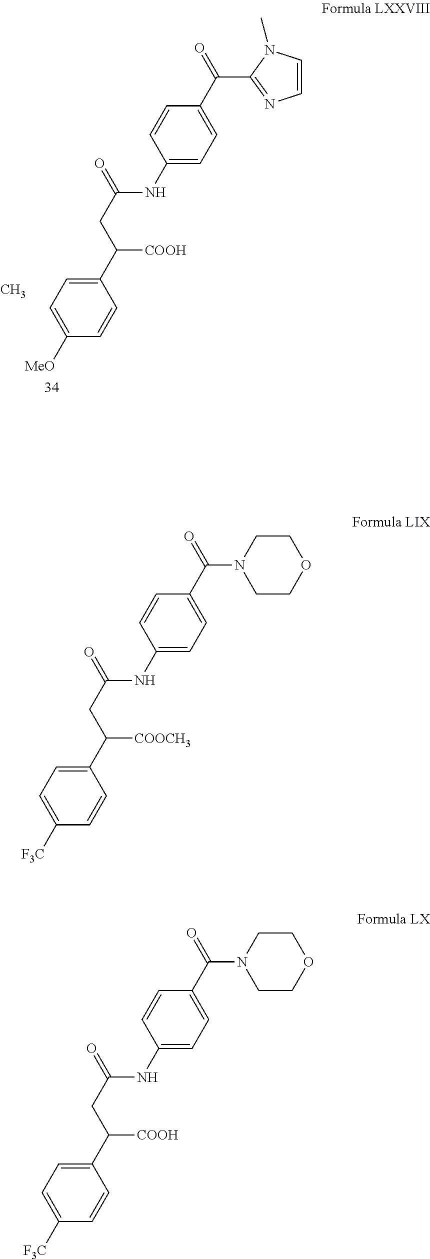

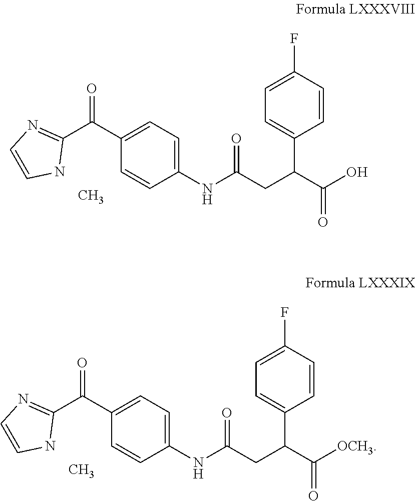

In certain particular (but non-limiting) embodiments, the Linker of Formula(s) I, II, and/or III comprises an amide group and a ring, wherein the Ring and R₂ form a cyclized system. Non-limiting examples of structures of compounds of the present disclosure that have such a linker include those having a general structure represented by Formula XXVII:

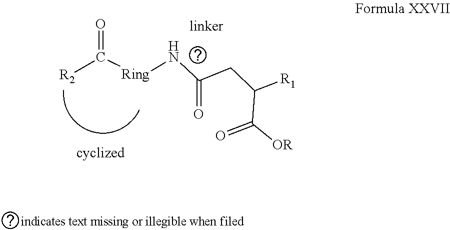

wherein the Ring comprises a 3-7 membered carbocyclic, alicyclic, heterocyclic, aromatic, or heteroaromatic ring. Particular (but non-limiting) examples of structures that meet Formula XXVII include structures represented by Formulas XXVIII, XXIX, and XXX:

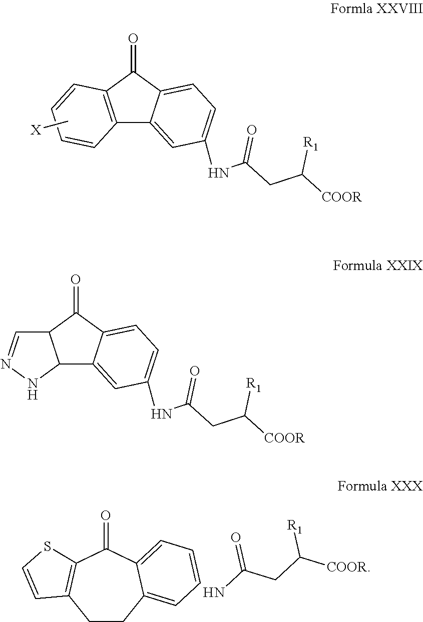

In certain particular (but non-limiting) embodiments, the Linker of Formula(s) I, II, and/or III comprises an amide group, and R₂ comprises a disubstituted cyclohexyl ring. Non-limiting examples of structures of compounds of the present disclosure that have such a linker include those having a general structure represented by Formula XXXII:

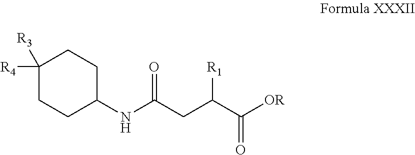

wherein each of R₃ and R₄ is selected from the group consisting of an alkyl group; an alkyl amine group; a hydroxyl group; a cyano group; a carboxylic acid; an aromatic, heteroaromatic, carbocyclic, or alicyclic ring incorporating at least one heteroatom; and combinations thereof. Particular (but non-limiting) examples of structures that meet the general structure of Formula XXXII include structures represented by Formulas XXXIII and XXXIV:

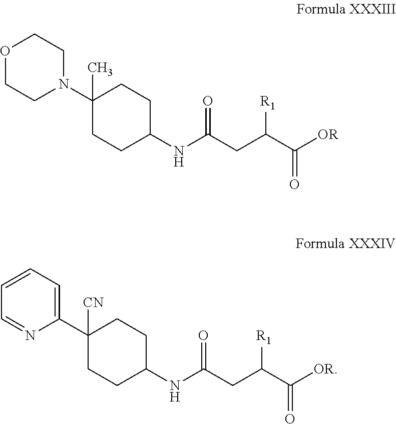

Certain non-limiting embodiments of the present disclosure are directed to a composition that comprises at least one LDHA-inhibitory compound that has a general structure represented by Formula XXXV:

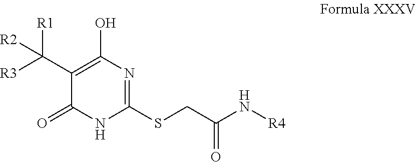

or an enantiomer, stereoisomer, or pharmaceutically acceptable salt thereof. In Formula XXXV, the R₄ group is defined as being selected from an alkyl group, a phenyl group, a substituted phenyl group, an aromatic ring, a heteroaromatic ring, a bicyclic group, a tricyclic group, a 3-7 membered alicyclic ring incorporating at least one heteroatom, a carbocyclic ring, a disubstituted carbocyclic ring, and combinations thereof. In addition, the R₁, R₂, and R₃ groups of Formula XXXV are defined as in one or (a) or (b): (a) R₁ is selected from an alkyl group, an aromatic ring, a substituted aromatic ring, a heteroaromatic ring, a carbocyclic ring, or an alicyclic ring incorporating at least one heteroatom; R₂ is selected from H, an alkyl group, or a cycloalkyl group; and R₃ is H; or (b) R₁, R₂ and R₃ are linked together to form an aromatic ring, a heteroaromatic ring, a carbocyclic ring, or an alicyclic ring comprising at least one heteroatom.

Non-limiting examples of structures that are included in Formula XXXV include Formulas XXXVII, XXXVIII, XXXIX, and LXIV:

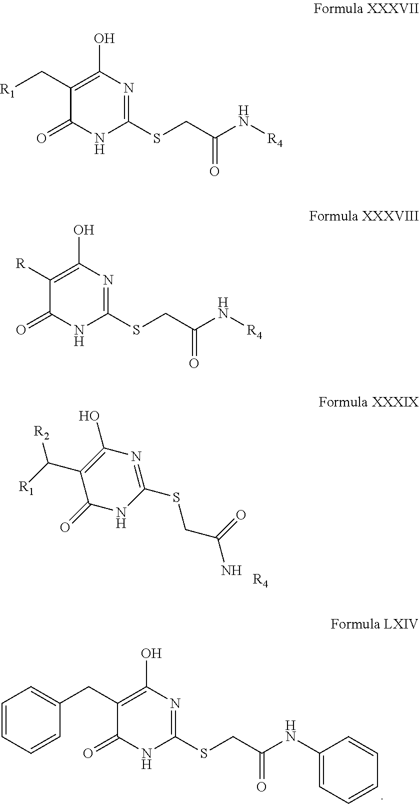

Certain non-limiting embodiments of the present disclosure are directed to a composition that comprises at least one LDHA-inhibitory compound that has a structure represented by Formula XL, XLI, XLII, or XLIII:

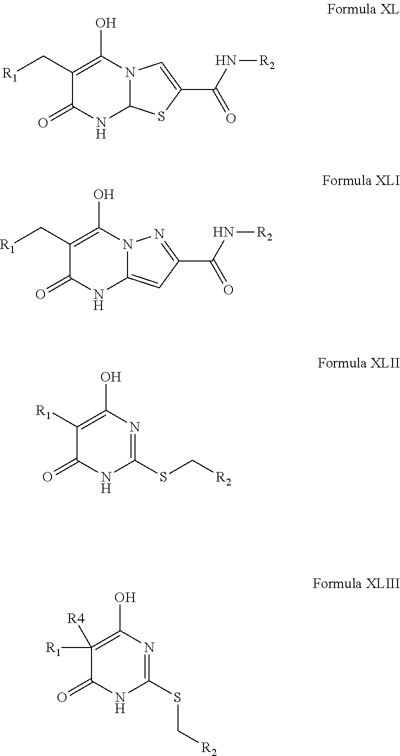

or an enantiomer, stereoisomer, or pharmaceutically acceptable salt thereof. In Formulas XL, XLI, XLII, and XLIII, each of the R₁, R₂, R₃, and R₄ groups is independently selected from an alkyl group, a phenyl group, a substituted phenyl group, an aromatic ring, a heteroaromatic ring, a bicyclic group, a tricyclic group, a 3-7 membered alicyclic ring incorporating at least one heteroatom, a carbocyclic ring, a disubstituted carbocyclic ring, and combinations thereof.

In certain non-limiting embodiments, any of the LDHA-inhibitory compositions disclosed or otherwise contemplated herein selectively inhibits LDHA at a half maximal inhibitory concentration (IC₅₀) of less than about 25 μmol/L (i.e., 25 μM), such as, but not limited to, less than about 20 μM, less than about 15 μM, about 10 μM, about 5 μM, about 1 μM, about 0.5 μM, about 0.1 μM, about 50 nM, about 40 nM, about 30 nM, about 20 nM, about 10 nM, about 1 nM, or the like. In a particular (but non-limiting) embodiment, the LDHA-inhibitory composition selectively inhibits LDHA at a half maximal inhibitory concentration (IC₅₀) that falls within a range formed of two of the above values (i.e., a range of from about to about 1 nM to about 25 μM, a range of from about 10 nM to about 20 μM, a range of from about 0.01 μM to about 10 μM, etc.).

In certain non-limiting embodiments, any of the LDHA-inhibitory compositions disclosed or otherwise contemplated herein selectively inhibits cell viability at a half maximal inhibitory concentration (IC₅₀) of less than about 100 μmol/L (i.e., 100 μM), such as, but not limited to, less than about 90 μM, about 80 μM, about 70 μM, about 60 μM, about 50 μM, about 45 μM, about 40 μM, about 35 μM, about 30 μM, about 25 μM, 20 μM, less than about 15 μM, about 10 μM, about 5 μM, about 1 μM, about 0.5 μM, about 0.1 μM, about 50 nM, about 40 nM, about 30 nM, about 20 nM, about 10 nM, about 1 nM, or the like. In a particular (but non-limiting) embodiment, the LDHA-inhibitory composition selectively inhibits cell viability at a half maximal inhibitory concentration (IC₅₀) that falls within a range formed of two of the above values (i.e., a range of from about 1 nM to about 50 μM, a range of from about 1 μM to about 30 μM, etc.).

The composition comprising at least one LDHA inhibitor may be provided with any formulation known in the art or otherwise contemplated herein. In certain particular (but non-limiting) embodiments, the composition comprising the at least one LDHA inhibitor contains one or more pharmaceutically acceptable carriers (and as such, the composition may also be referred to as a “pharmaceutical composition”). Non-limiting examples of suitable pharmaceutically acceptable carriers include water; saline; dextrose solutions; fructose or mannitol; calcium carbonate; cellulose; ethanol; oils of animal, vegetative, or synthetic origin; carbohydrates, such as glucose, sucrose, or dextrans; antioxidants, such as ascorbic acid or glutathione; chelating agents; low molecular weight proteins; detergents; liposomal carriers; buffered solutions, such as sodium chloride, saline, phosphate-buffered saline, and/or other substances which are physiologically acceptable and/or safe for use; diluents; excipients such as polyethylene glycol (PEG); or any combination thereof. Suitable pharmaceutically acceptable carriers for pharmaceutical formulations are described, for example, in Remington: The Science and Practice of Pharmacy, 23rd ed (2020).

In certain particular (but non-limiting) embodiments, the composition comprising the LDHA inhibitor(s) may further contain one or more additional active agents. Various active agents utilized alone or in combination with existing LDHA inhibitor(s) to treat cancer are known in the art. Non-limiting examples of additional therapeutic agents that can be utilized in accordance with the present disclosure in combination with LDHA inhibitor(s) include an oxidative phosphorylation (OXPHOS) inhibitor, an MTORC1/TORC2 inhibitor, an autophagy inhibitor, a chemotherapeutic drug, and the like as well as any combinations thereof. Particular non-limiting examples of additional therapeutic agents that can be utilized in accordance with the present disclosure include metformin, phenformin, gemcitabine, rapamycin, an analog of any of the foregoing, and combinations thereof.

In certain particular (but non-limiting) embodiments, the LDHA inhibitor(s) present in the composition is conjugated to another substance. For example, but not by way of limitation, the LDHA inhibitor(s) may be conjugated to a particle or other substance for targeted delivery of the drug to a specific location in the body. In another particular (but non-limiting) embodiment, the composition may comprise LDHA inhibitor(s) encapsulated in a liposomal formulation or nanoparticle.

In addition, any of the compositions of the present disclosure may contain other agents that allow for administration of the compositions via a particular administration route. For example, but not by way of limitation, the compositions may be formulated for administration by oral, topical, transdermal, parenteral, subcutaneous, intranasal, mucosal, intramuscular, intraperitoneal, intravitreal, and/or intravenous routes. Based on the route of administration, the compositions may also contain one or more additional components in addition to the active agent. Examples of additional secondary compounds that may be present include, but are not limited to, fillers, salts, buffers, preservatives, stabilizers, solubilizers, wetting agents, emulsifying agents, dispersing agents, and other materials well known in the art.

Certain non-limiting embodiments of the present disclosure are directed to compositions that contain mixtures of two or more of any of the LDHA inhibitor compounds disclosed or otherwise contemplated herein. For example, but not by way of limitation, the composition may contain a mixture of about two, about three, about four, about five, about six, about seven, about eight, about nine, about ten, about 11, about 12, about 13, about 14, about 15, about 16, about 17, about 18, about 19, about 20, or more of the LDHA inhibitor compounds disclosed or otherwise contemplated herein, as well as a range of two of any of the values above (i.e., a mixture comprising a range of from about two to about 20 compounds, etc.).

Certain non-limiting embodiments of the present disclosure are directed to a method of synthesizing any of the LDHA-inhibitory compounds as described or otherwise contemplated herein. Said synthesis methods may include one or more steps as described in the example section or as otherwise contemplated herein.

Each of the synthesis methods described or otherwise contemplated herein may produce the LDHA inhibitor compounds with any level of yield. For example (but not by way of limitation), the compounds can be synthesized with a yield of at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, and at least about 99%. In addition, the scope of the presently disclosure also includes the production of the compounds at any percent yield that falls within a range formed from the combination of two values listed above (for example, a range of from about 1% to about 99%, a range of from about 10% to about 98%, a range of from about 30% to about 97%, a range of from about 50% to about 96%, a range of from about 60% to about 95%, etc.).

Certain non-limiting embodiments of the present disclosure are directed to an LDHA-inhibitory compound produced by any of the methods described or otherwise contemplated herein.

Certain non-limiting embodiments of the present disclosure are directed to a pharmaceutical composition that comprises at least one of any of the LDHA inhibitor compounds disclosed or otherwise contemplated herein. In addition, the pharmaceutical composition comprises a pharmaceutically acceptable carrier (as defined in detail herein above) and may be formulated in any manner disclosed or otherwise contemplated herein.

Certain non-limiting embodiments of the present disclosure are directed to a method of utilizing any of the compositions/pharmaceutical compositions disclosed or otherwise contemplated herein to inhibit LDHA expression and/or activity and/or to treat various conditions, diseases, or disorders in which LDHA activity is increased.

Certain non-limiting embodiments of the present disclosure are directed to a method of inhibiting LDHA expression and/or enzymatic activity in at least one cell. The method comprises exposing the at least one cell to at least one of any of the compositions disclosed or otherwise contemplated herein.

Certain non-limiting embodiments of the present disclosure are directed to a method of reducing viability of cancer cells. The method comprises administering to the cancer cells at least one of any of the compositions disclosed or otherwise contemplated herein.

Certain non-limiting embodiments of the present disclosure are directed to a method that comprises administering at least one of any of the compositions disclosed or otherwise contemplated herein to a subject in need thereof. The subject may be suffering from or predisposed to any condition, disease, or disorder in which LDHA activity is increased. In certain particular (but non-limiting) embodiments, the subject has cancer.

The composition may be administered to the subject by any route known in the art or otherwise contemplated herein. Non-limiting examples of administration routes include oral, topical, transdermal, parenteral, subcutaneous, intranasal, mucosal, intramuscular, intraperitoneal, intravitreal, and/or intravenous routes.

The methods of the present disclosure may be utilized to treat any types of cancer cells/cancers/tumors that will respond to treatment with LDHA inhibitor(s). Non-limiting examples of cancer cells/cancers/tumors that can be treated in accordance with the present disclosure include carcinomas (such as, but not limited to, breast, lung, cerebral, bladder, thyroid, prostate, colon, rectum, pancreas, stomach, liver, uterine, hepatic, renal, prostate, cervical, and ovarian carcinomas); lymphomas (such as, but not limited to, Hodgkin and non-Hodgkin lymphomas); neuroblastomas; melanomas; myelomas; Wilm's tumors; leukemias (such as, but not limited to, acute lymphoblastic leukemias and acute myeloblastic leukemias); astrocytomas; gliomas; retinoblastomas; and the like, as well as any combinations thereof.

The composition comprising LDHA inhibitor(s) may be administered to the cells/subject at any concentration that provides a therapeutically effective concentration of the LDHA inhibitor(s). The therapeutically effective concentration of LDHA inhibitor(s) utilized in accordance with the present disclosure may be, for example (but not by way of limitation), about 1 nM, about 2 nM, about 3 nM, about 4 nM, about 5 nM, about 6 nM, about 7 nM, about 8 nM, about 9 nM, about 10 nM, about 11 nM, about 12 nM, about 12.5 nM, about 13 nM, about 14 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 75 nM, about 80 nM, about 85 nM, about 90 nM, about 95 nM, about 100 nM, about 110 nM, about 120 nM, about 130 nM, about 140 nM, about 150 nM, about 200 nM, about 250 nM, about 300 nM, about 350 nM, about 400 nM, about 450 nM, about 500 nM, about 550 nM, about 600 nM, about 650 nM, about 700 nM, about 750 nM, about 800 nM, about 850 nM, about 900 nM, about 950 nM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 31 μM, about 32 μM, about 33 μM, about 34 μM, about 35 μM, about 36 μM, about 37 μM, about 38 μM, about 39 μM, about 40 μM, about 41 μM, about 42 μM, about 43 μM, about 44 μM, about 45 μM, about 46 μM, about 47 μM, about 48 μM, about 49 μM, about 50 μM, about 55 μM, about 60 μM, about 65 μM, about 70 μM, about 75 μM, about 80 μM, about 85 μM, about 90 μM, about 95 μM, about 100 μM, and the like, as well as a range formed from any of the above values (e.g., a range of from about 10 nM to about 100 μM, a range of from about 10 nM to about 50 μM, etc.).

In certain particular (but non-limiting) methods, the at least one composition selectively inhibits cell viability at a half maximal inhibitory concentration (IC₅₀) of less than about 100 μmol/L (i.e., 100 μM), such as, but not limited to, less than about 90 μM, about 80 μM, about 70 μM, about 60 μM, about 50 μM, about 45 μM, about 40 μM, about 35 μM, about 30 μM, about 25 μM, 20 μM, less than about 15 μM, about 10 μM, about 5 μM, about 1 μM, about 0.5 μM, about 0.1 μM, about 50 nM, about 40 nM, about 30 nM, about 20 nM, about 10 nM, about 1 nM, or the like. In a particular (but non-limiting) embodiment, the at least one composition selectively inhibits cell viability at a half maximal inhibitory concentration (IC₅₀) that falls within a range formed of two of the above values (i.e., a range of from about 50 μM to about 1 nM, a range of from about 30 μM to about 1 μM, etc.).

In certain particular (but non-limiting) embodiments, any of the methods described or otherwise contemplated herein may further include the step of administering at least one additional composition (containing at least one additional active agent) to the cell(s)/subject. Various active agents utilized alone or in combination with existing LDHA inhibitor(s) to treat cancer are known in the art. Non-limiting examples of therapeutic agents that can be utilized in accordance with the present disclosure in a concurrent therapy with LDHA inhibitor(s) include an oxidative phosphorylation (OXPHOS) inhibitor, an MTORC1/TORC2 inhibitor, an autophagy inhibitor, a chemotherapeutic drug, and the like as well as any combinations thereof. Particular non-limiting examples of additional therapeutic agents that can be utilized in accordance with the present disclosure include metformin, phenformin, gemcitabine, rapamycin, an analog of any of the foregoing, and combinations thereof.

In addition, the at least one additional composition may be administered substantially simultaneously or wholly or partially sequentially with the at least one LDHA-inhibitory composition. When administered sequentially, the at least one additional composition may be administered before or after the LDHA-inhibitory composition(s).

Any of the steps of the methods of the present disclosure may be repeated one or more times. Each of the steps can be repeated as many times as necessary. The steps of administering compositions/additional therapies may be repeated various times and at various intervals to follow any known and/or generally accepted dosage/treatment regimen for the composition(s)/therapy(ies) or similar substances.

Certain non-limiting embodiments of the present disclosure are directed to kits that include one or more of any of the compositions/pharmaceutical compositions disclosed or otherwise contemplated herein. The kits may optionally further include one or more of any of the optional compositions disclosed or otherwise contemplated herein (such as, but not limited to, one or more optional compositions containing at least one additional active agent). The kits may optionally further include one or more devices utilized in the administration steps.

In a particular (but non-limiting) embodiment, the kit may further include instructions for performing any of the methods disclosed or otherwise contemplated herein. For example (but not by way of limitation), the kit may include instructions for when and how to administer the composition comprising LDHA inhibitor(s) and optionally how to administer one or more optional additional compositions in relation to the administration of the composition comprising LDHA inhibitor(s) and/or administration of one or more optional therapy steps.

In addition to the components described in detail herein above, the kits may further contain other component(s)/reagent(s) for performing any of the particular methods described or otherwise contemplated herein. The nature of these additional component(s)/reagent(s) will depend upon the particular treatment format, and identification thereof is well within the skill of one of ordinary skill in the art; therefore, no further description thereof is deemed necessary. Also, the components/reagents present in the kits may each be in separate containers/compartments, or various components/reagents can be combined in one or more containers/compartments, depending on the sterility, cross-reactivity, and stability of the components/reagents.

The kit may be disposed in any packaging that allows the components present therein to function in accordance with the present disclosure. In addition, the kit can further include a set of written instructions explaining how to use one or more components of the kit. A kit of this nature can be used in any of the methods described or otherwise contemplated herein.

### EXAMPLE

An Example is provided hereinbelow. However, the present disclosure is to be understood to not be limited in its application to the specific experimentation, results, and laboratory procedures disclosed herein. Rather, the Example is simply provided as one of various embodiments and is meant to be exemplary, not exhaustive.

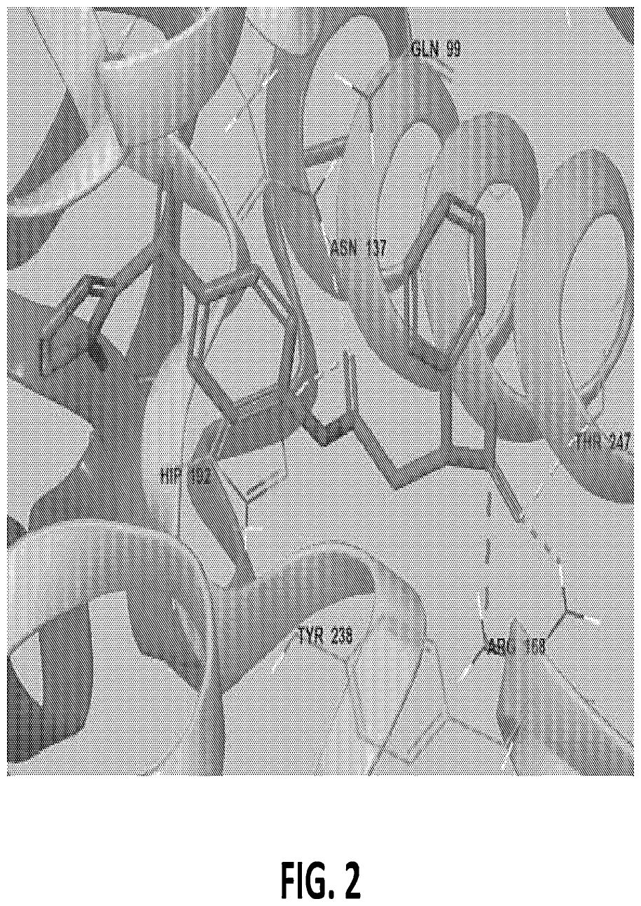

Molecular Modeling: Novel chemotypes incorporating succinic acid scaffold as lactate dehydrogenase-A (LDHA) inhibitors were identified using molecular modeling and virtual screening. Techniques utilized included an innovative 2D atom-based fingerprint, pharmacophore modeling, ensemble docking, and molecular dynamics simulations. A discovered compound, LDI-7 (Formula IX), containing a succinic acid monoxide moiety, was shown to possess potent LDHA inhibitory activity (LDHA IC₅₀ 0.117 μM). The docked pose of LDI-7 in the LDHA active site is shown in FIG. 2. LDI-7 also possessed anticancer activity and inhibited the viability of MIA PaCa-2 cells with IC₅₀ of 12.26 μM. Medicinal chemistry optimization was carried out to identify related compounds with LDHA inhibitory and cellular anticancer activity. Specifically, LDI-7, compound 5, and compound 17 (Formulas IX, XI, and XII, respectively) are representative (but non-limiting) lead LDHA inhibitors.

A novel compound LDI-22 (Formula XXXV) with a 4-hydroxy pyrimidinone scaffold was also discovered. LDI-22 inhibited LDHA with IC₅₀ of 9.45 μM. LDI-22 blocked lactate production in cells and inhibited the viability of three pancreatic cancer cell lines with IC₅₀ up to 14.64 μM against PANC-1 cells which, under normoxic conditions, is already comparable or more potent than most currently available LDHA inhibitors.

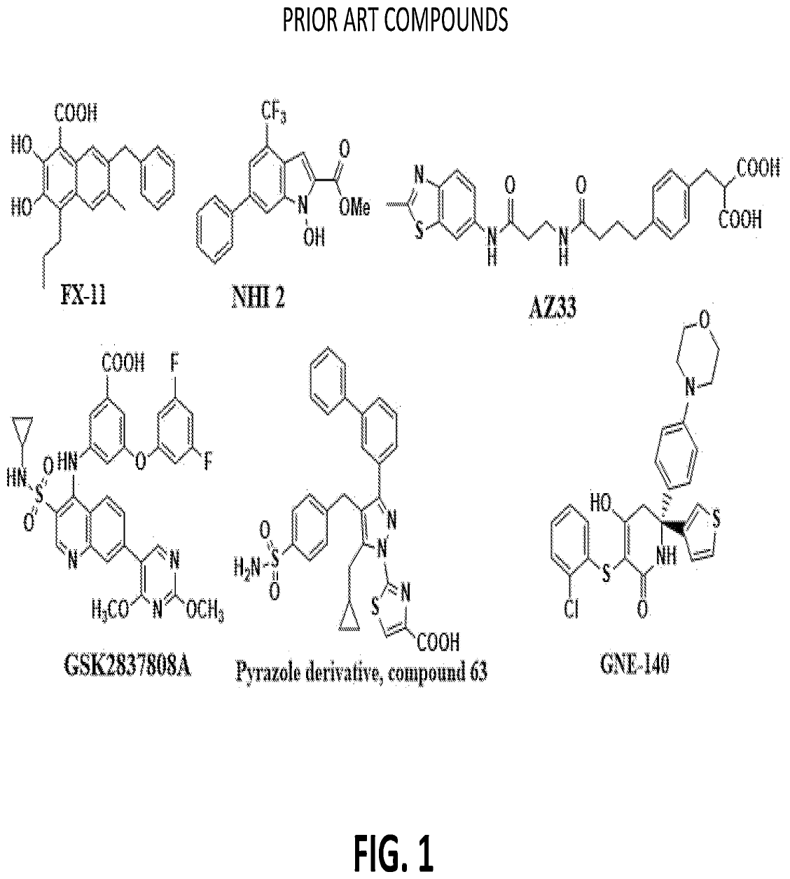

Biochemical LDHA inhibition assay: The hLDH5 (LDHA) enzymatic activity was measured by fluorescence (excitation@ 340 nm, emission@ 460 nm) to monitor the rate of conversion of NADH to NAD+at 37° C. NHI-2 and DMSO was used as a positive control and negative control, respectively. Notably, the compounds of the present disclosure are more potent than some licensed and commercially available selective LDHA inhibitors such as FX-11 and NHI-2 (FIG. 1). FX-11 showed a Ki of 8 μM for LDHA and is shown to inhibit the progression of human pancreatic and lymphoma cancer xenografts (Le et al. (2010) Proc Natl Acad Sci USA, 107(5):2037-42). NHI-2 displayed in vitro biochemical IC₅₀ of 14.7 μM for LDHA, induced apoptosis, and synergistically enhanced gemcitabine activity in PDAC cells (Granchi et al. (2013) Org Biomol Chem, 11(38):1-18; Maftouh et al. (2014) British Journal of Cancer, 110(1):172-72).

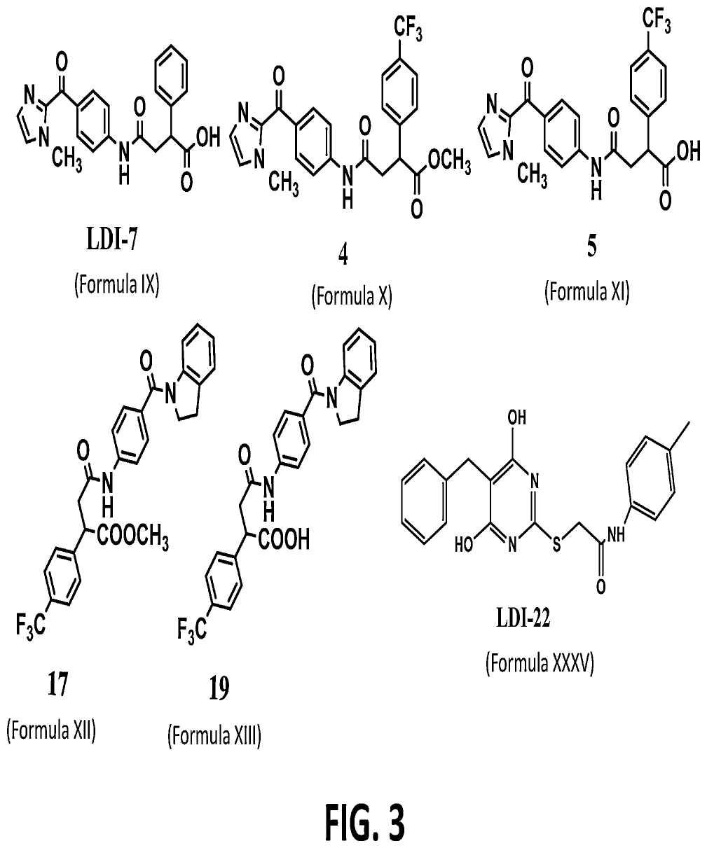

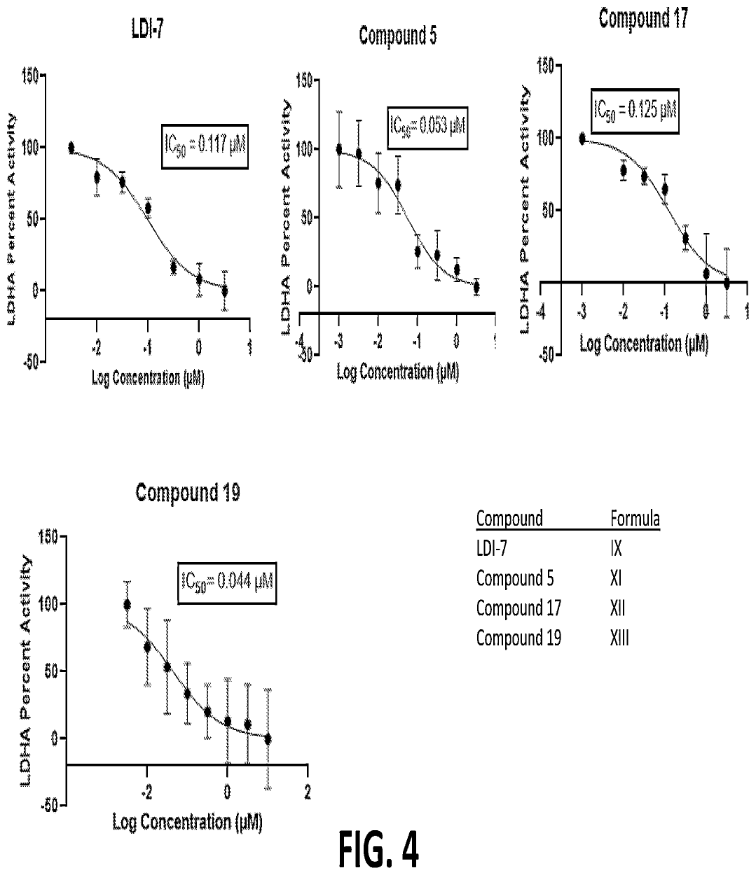

In contrast, the compounds of the present disclosure inhibited LDHA in low micromolar to up to nanomolar concentrations, with compound 5, LDI-7, and compound 17 (Formulas XI, IX, and XII, respectively) exhibiting potent inhibition of LDHA with IC₅₀s of 53 nM, 0.117 μM, and 0.125 μM, respectively (FIGS. 3-4). In addition, the biochemical potencies of representative compounds produced in accordance with the present disclosure are shown in Table 1.

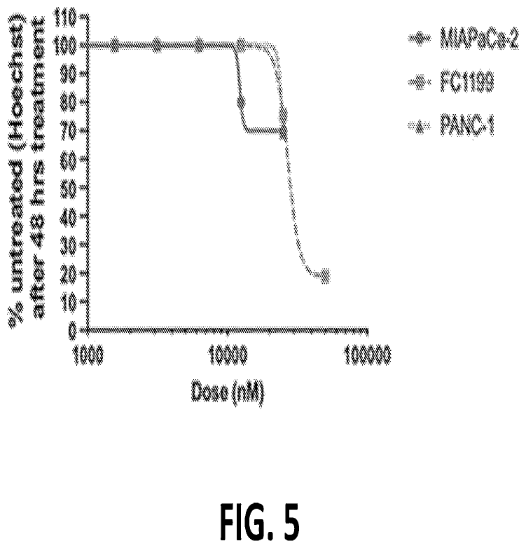

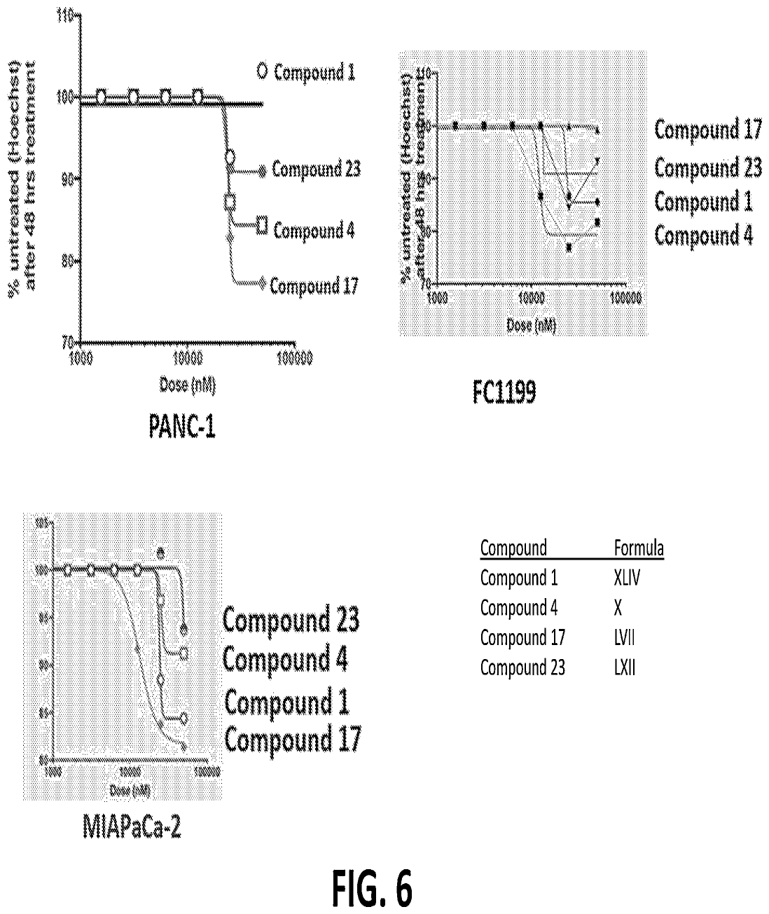

Cytotoxicity study: Anticancer potency was investigated using a fluorescence-based high throughput confocal microscopy cell viability/proliferation/cell death assay. The effects of the compounds of the present disclosure on cell viability was determined in two human pancreatic cancer cell lines, PANC-1 and MIA PaCa-2 (Table 1). Selected compounds were also tested in the FC1199 pancreatic cancer line from the KPC-1 genetically engineered mouse model. Compounds were also tested in hTERT immortalized normal pancreatic (HPNE) cell lines to rule out nonspecific cytotoxicity of compounds. The identified compounds selectively killed pancreatic cancer cells with IC₅₀ values in the low micromolar range without causing cytotoxicity in normal cells. The lead compounds were comparable or even more potent than reported licensed compounds FX-11 and NHI-2. For example, FX-11 showed an IC₅₀ of 23.3 μM in HeLa cells. NHI-2 inhibited the viability of PANC-1 and HeLa human cervical carcinoma cells with IC₅₀s of 22.2 μM and 33.4 μM, respectively, under normoxic conditions. The compounds of the present disclosure also inhibited the growth of pancreatic cancer cells in low micromolar levels (Table 1). The Dose response curves of selected compounds from the cytotoxicity assays are shown in FIGS. 5-6. Specifically, LDI-7 and compound 17 (Formulas IX and XII, respectively) inhibited the viability of MIA PaCa-2 cells with IC₅₀s of 12.2 μM and 13.3 μM, respectively. In addition, certain lead compounds' inhibition of cancer cell growth under normoxic conditions may be comparable to GSK2837808A and GNE-140 that showed IC₅₀s of 2.97 μM and 2.0 μM, under hypoxic conditions. As reported in the literature, the cytotoxicity of the compounds of the present disclosure improves if tested under hypoxic conditions, which induces LDHA and represents the conditions of the tumor microenvironment. The anticancer activity of compound 5 (Formula XI), which has the best IC₅₀ of 0.053 μM against LDHA, is evaluated in additional studies.

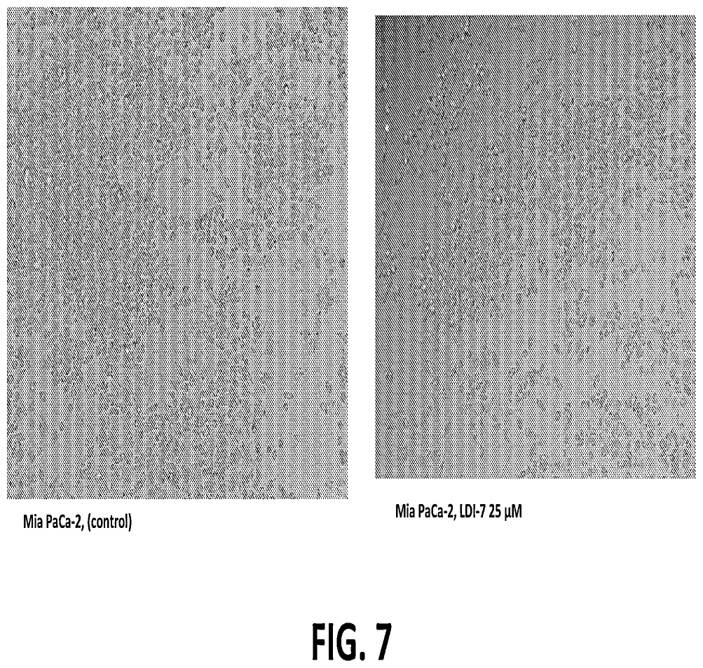

A representative example of a cell viability dose-response curve, which represents dose (M) vs % untreated (Hoechst) after 48 hours treatment, of compounds 1, 4, 17, and 23 (Formulas XLIV, X, LVII, and LXII, respectively) in PANC-1, FC1199, and MIAPaCa-2 cells is shown in FIG. 6. A representative example of morphological changes induced upon treatment with compound LDI-7 (Formula IX, 25 μM) in MIA PaCa-2 cells is shown in FIG. 7.

LDI-22 (Formula XXXV) is a structurally different compound containing a hydroxypyrimidinone ring. It also inhibits LDHA and exhibits cytotoxicity against pancreatic cancer cell line in low μM concentrations.

TABLE 1
Biochemical Potency and Cytotoxicity of LDHA-Inhibitory Compounds

| Biochemical potency | | | | | |
|---|---|---|---|---|---|
| LDHA inhibition IC₅₀ ± SD (μM) | | Cytotoxicity IC₅₀ ± SD (μM) | | | |
| Cmpd | LDHA | PANC-1 | MIA PaCa-2 | FC-1199 | HPNE Normal |
| LDI-7 | 0.117 ± 0.028 | 24.75 ± 2.28 | 12.26 ± 1.12 | 27.09 ± 3.06 | >50 |
| 1 | 14.15 ± 3.85 | >50 | 24.213 ± 3.4 | 23.374 ± 3.5 | 26.458 ± 4.4 |
| 4 | 4.89 ± 1.65 | 23.944 ± 1.8 | 39.106 ± 2.917 | 12.121 ± 1.2 | 49.471 ± 7.3 |
| 5 | 0.053 ± 0.02 | 43.31 ± 3.98 | 19.610 ± 2.216 | 20.827 ± 2.97 | >50 |
| 17 | 0.125 ± 0.044 | 24.193 ± 1.08 | 13.320 ± 2.2 | 73.357 ± 6.8 | 50.671 ± 3.6 |
| 19 | 0.044 ± 0.008 | 43.07 ± 4.87 | 20.380 ± 1.875 | 56.533 | >50 |
| 20 | 18.00 ± 1.61 | >50 | 12.41 ± 1.4 | >50 | >50 |
| 21 | 8.6 ± 2.21 | >50.0 | 15.50 ± 2.36 | >50 | >50 |
| 23 | 5.58 ± 1.05 | 22.256 ± 2.0 | 40 ± 9.23 | 13.338 ± 0.9 | >50 |
| 24 | 18.71 ± 0.22 | >50 | 12.11 ± 1.04 | >50 | >50 |
| 25 | 15.28 ± 1.57 | 46.039 ± 3.2 | 20.236 ± 3.0 | 49.431 ± 8.1 | 27.956 ± 2.2 |
| LDI-22 | 9.45 ± 1.83 | 14.64 ± 0.89 | 25.851 ± 8.212 | 12.613 ± 1.292 | >50 |

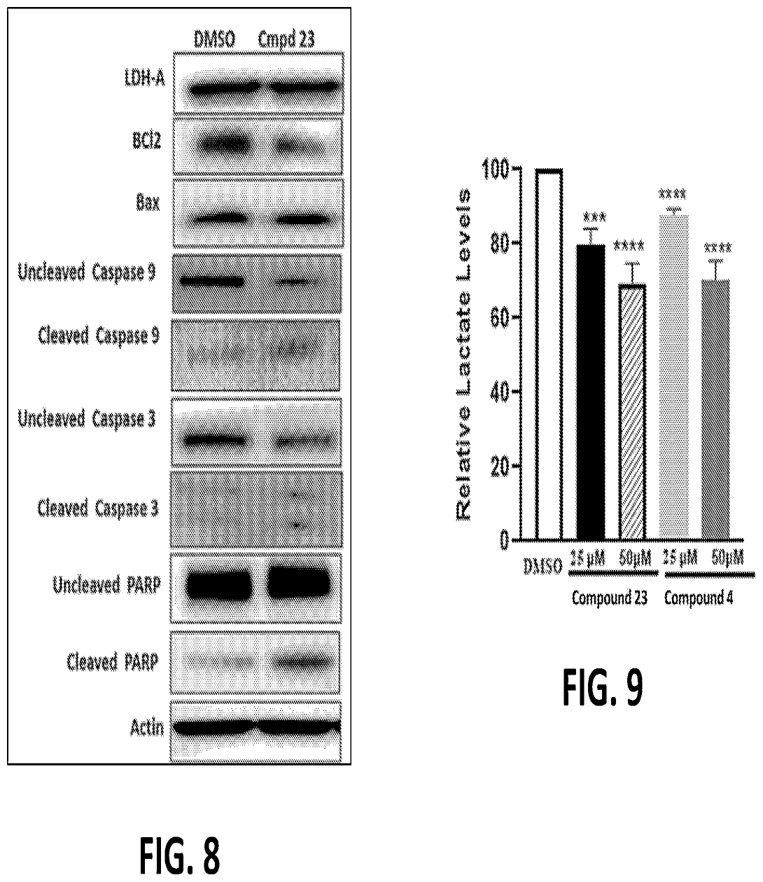

Western Blotting: FIG. 8 shows Western blot analysis of apoptotic proteins in MIA PaCa-2 cells treated with compound 23 (Formula LXII) for 24 h. Compound 23 (Formula XIII) was found to inhibit the activity of LDHA without affecting its expression. Western blot analysis revealed activation of proapoptotic proteins, like cleaved poly (ADP-ribose) polymerase (PARP), cleaved Caspase 9, and cleaved Caspase 3. In contrast, the expression of the anti-apoptotic protein, B-cell lymphoma 2 (Bcl-2), decreased along with a decrease in the BCL2/BAX expression ratio. While not wishing to be bound by any particular theory, these results indicate that compound 23 may activate the intrinsic pathway of apoptosis, a mechanism of cell death.

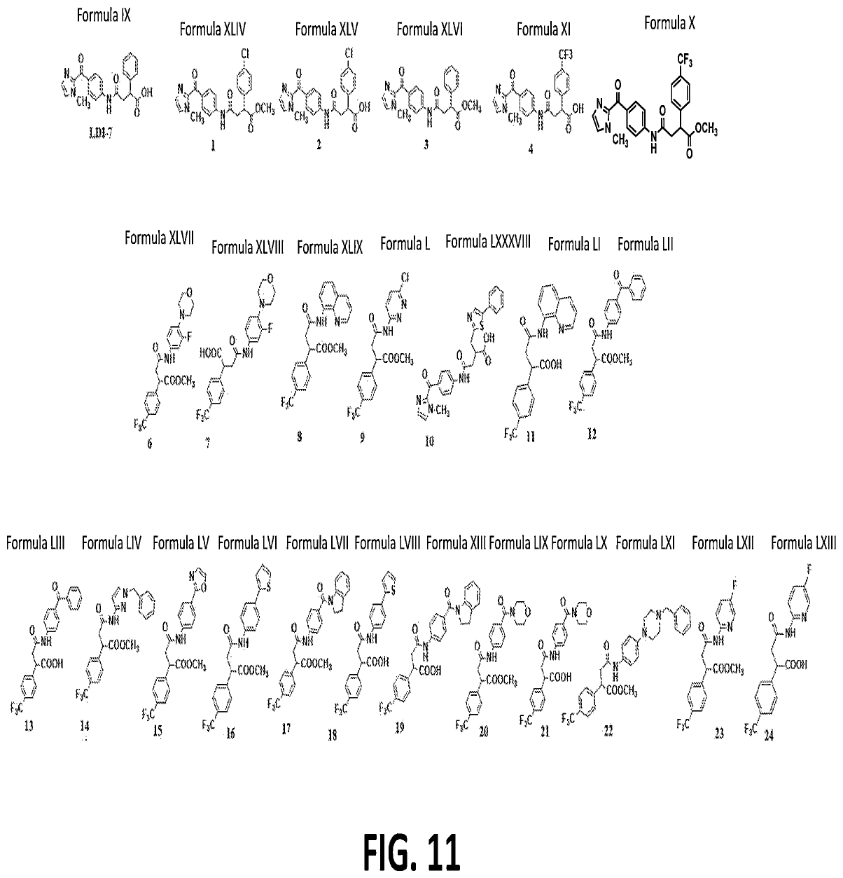

Inhibition of lactate production in cells: Lactate production is regarded as a key event involved in carcinogenesis and immune escape. MIA PaCa-2 cells were treated with LDI-7 and LDI-22 (Formulas IX and XXXV, respectively), and lactate that accumulated in the cell culture was determined after 6 hours. Both compounds demonstrated inhibition of lactate production in a dose-dependent manner. The compounds were tested in varied nutrient conditions. Initially, regular DMEM medium (25 mM Glucose, 2 mM Glutamine, and 1 mM Pyruvate) was used (FIG. 10, Panel A). Next, compounds were evaluated in nutrient-stressful conditions with low glucose (10 mM) and no pyruvate in the cell culture media (FIG. 10, Panel B). LDI-7 and LDI-22 resulted in about a 30%-50% reduction in lactate levels when treated with 15 μM and 30 μM concentrations, respectively. The lactate levels were normalized to protein concentration, measured by a BCA protein assay kit, and expressed as a percentage relative to control.

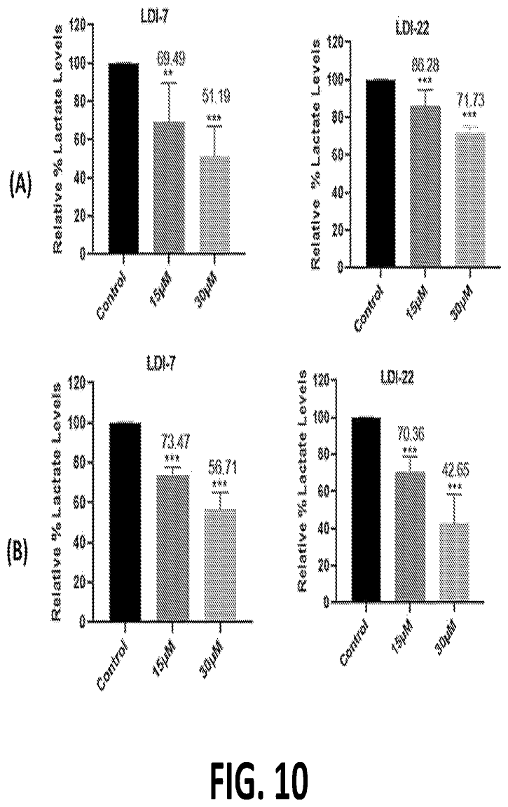

In addition, FIG. 9 demonstrates that compounds 23 and 4 inhibit lactate production in MIA PaCa-2 cells in a dose-dependent manner. MIA PaCa-2 cells were treated with indicated concentrations of compounds or untreated control for six hours in DMEM containing 25 mM Glucose, 2 mM glutamine, and 1 mM pyruvate. The lactate accumulated in the cell culture was determined by Lactate Assay Kit (BioVision, Mountain View, CA, USA).

### Experimental Section

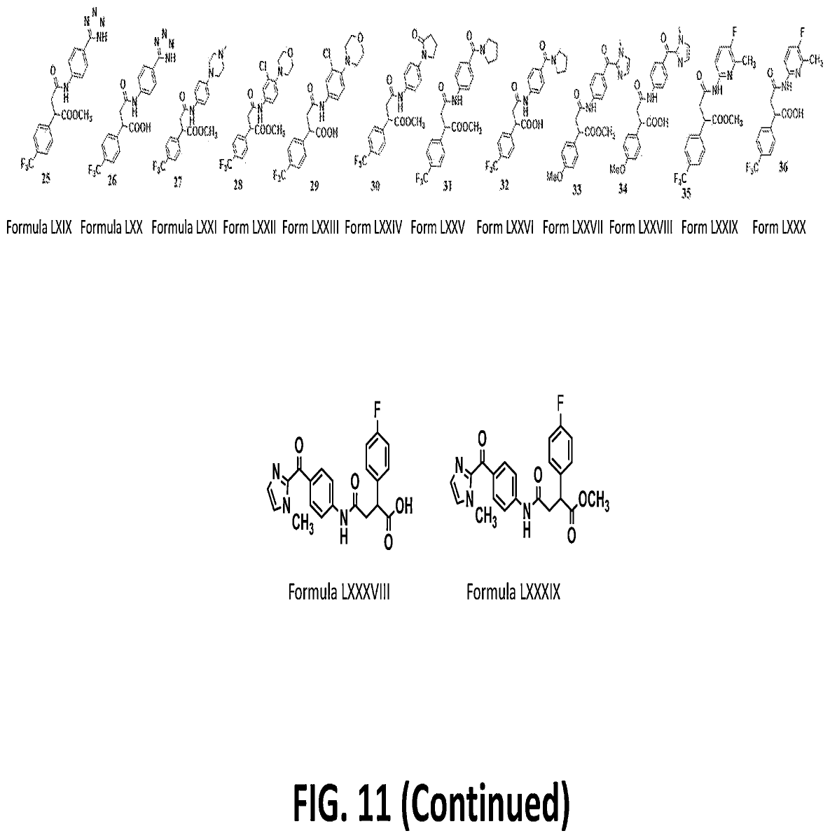

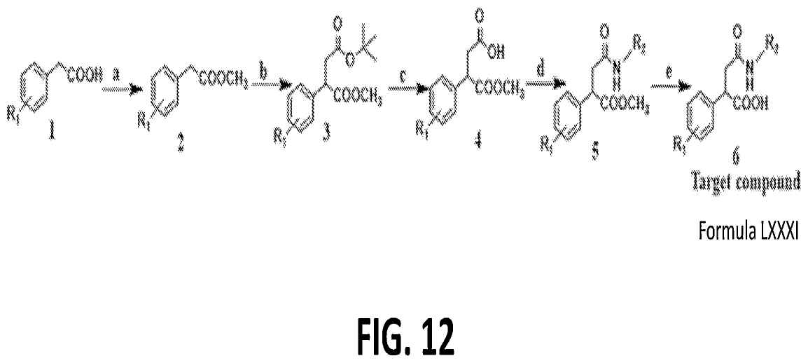

Synthesis: Compounds shown in the FIG. 11 were synthesized via the schemes shown in FIG. 12 and characterized using 1H NMR and mass spectroscopy. Final compounds were also characterized by melting points and high-resolution mass spectroscopy (HRMS). The purity of the final compounds was determined using HPLC. Medicinal chemistry optimization and synthesis of enantiomerically pure compounds is carried out iteratively to develop more potent compounds in the series. The synthesis of LDI-7 and related analogs is shown in FIG. 12. It involves converting phenyl acetic acid starting materials to their corresponding methyl esters, intermediate 2. The next step involves a nucleophilic substitution with tert-butyl bromoacetate to give diester derivative 3, which is selectively hydrolyzed to yield intermediate 4. The final two steps involve coupling with diverse amines, followed by hydrolysis of the methyl esters to give target compounds 6. Note that these compounds have one chiral center and were tested as a racemic mixture. To evaluate the stereogenic center, asymmetric synthesis is carried out to yield enantiomerically pure target compounds.

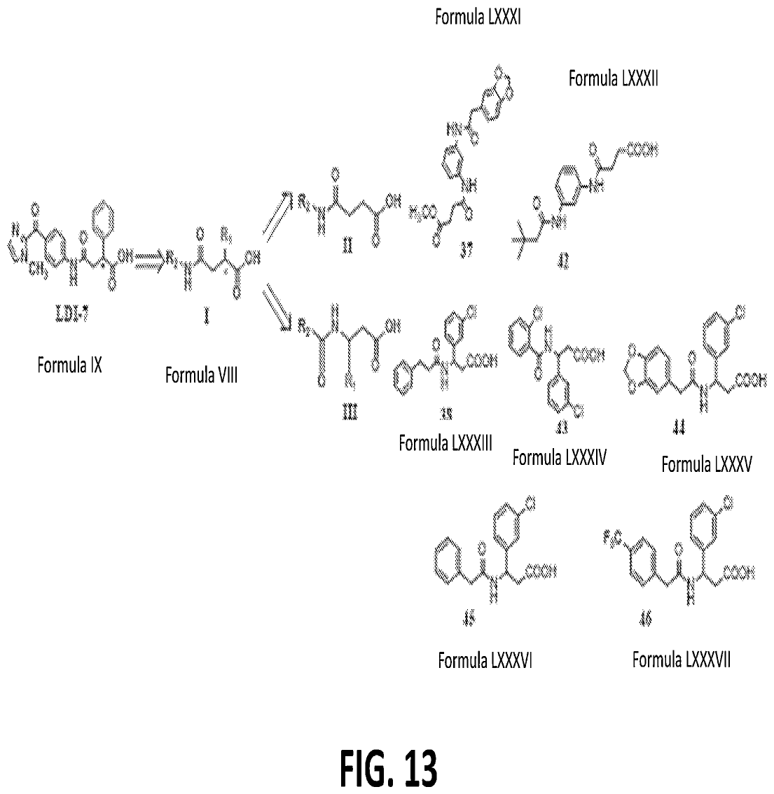

In addition, FIG. 13 shows the structural modification of LDI-7 (i.e., Formula VIII) to arrive at general Formulas II and III. Formula II does not have the R₁ group on the succinic acid monoamide moiety. Formula III has the reverse amide, and the R₁ group is on the adjacent carbon. Structures LXXXI-LXXXVII represent examples of formulas II and III.

LDH inhibition assay. The hLDH5 (LDHA) inhibition assay was performed by measuring the fluorescence (excitation@ 340 nm, emission@ 460 nm) and monitoring the rate of conversion of NADH to NAD+at 37° C. as reported. The apparent Michaelis Menten constant (Km) of NADH for hLDH5 was determined using 0.003 units of hLDH5 per well under saturated pyruvate (1440 μM) and increasing NADH concentrations from 12.5 μM-150 μM in 100 mM sodium phosphate buffer (pH 7.4). Michaelis Menten constants were determined with non-linear regression analysis using GraphPad Prism 9.0. The above conditions were used for the hLDH5 inhibition assays, which were carried out in 96-well plates with the following final enzyme and buffer concentration: 100 mM phosphate buffer (pH=7.4), 0.003 Units of LDHA, 40 M NADH (˜2×Km), and 1.44 mM pyruvate (saturated pyruvate conditions). The stock solution of compounds was prepared in DMSO. NHI-2 and DMSO were used as positive and negative controls, respectively. The experiment involved adding a solution of compounds in DMSO to the enzyme and NADH in a phosphate buffer. The assay plate was incubated at 25° C. for 10 minutes, and a baseline read was taken, after which pyruvate was added. The fluorescence was read for 5 minutes every 30 seconds in a microplate reader. The slope of a suitable linear timeframe was calculated with the curve bottom assigned to the initial 5-second recording before adding pyruvate (background rate) and the curve top to the negative (DMSO only) control wells rate. IC50 values were determined from dose-response curves using four-parameter logistic non-linear regression analysis in Prism Software v9.0. The assays were performed in triplicates and the data is presented as mean±SD (n=3). The Km for hLDH1 was determined with 0.003 units of hLDH5 using saturated pyruvate and increasing NADH concentrations (5 μM-150 μM). The hLDH1 inhibitory activity of promising hits was determined using similar assay conditions with 0.005 units of hLDH1, 1440 μM pyruvate, and 40 μM NADH (˜2×Km) concentrations,

Cell lines. The human PANC-1, MiaPaCa-2, and HPNE cell lines were obtained from American Type Culture Collection (ATCC, Manassas, VA, USA). Mouse pancreatic cancer FC1199 cells were obtained from the KPC-1 genetically engineered mouse model that has mutated K-Ras, metastasizes to the liver, and forms desmoplasia. All cell lines were cultured in DMEM (Hyclone, DMEM/High Glucose, Cat# SH30243.01 with the addition of 10% Cosmic Calf Serum) (GIBCO, DMEM/High Glucose, DMEM Cat# 11995040 with 10% FBS) and Pen/Strep and maintained in a 37° C., 5% CO₂/95% humidified air incubator.

Cytotoxicity assay. Cytotoxicity was assessed using the viability/proliferation/necrosis assay. For this assay, cancer cells were plated on a 96-well plate, and 1:2 serial dilution of compounds was added with 25 μM as the maximum dose for 48 hr. At the end of 48 hr period, Hoechst 33342 (1.0 μM) and SYTOX Green (0.5 μM) fluorescent dyes were added to each well for 15 min. Confocal images were acquired using the Operetta High Content Imaging System (PerkinElmer). In each well, five fields are screened using a 10×field objective with Hoechst 33342 detected using an excitation wavelength of 360-400 nm and an emission wavelength of 490-500 nm. SYTOX Green will be detected using an excitation wavelength of 500-520 nm and an emission wavelength of 520-530 nm. Brightfield images will also be acquired for each field. Images will be analyzed using Harmony software (PerkinElmer) with the cell-permeable vital dye Hoechst 33342 to identify cell nuclei (i.e., for cell counts), while the normally cell impermeable SYTOX Green will be used to identify necrotic cells. Cell counts were summed over the five fields for each well, and the percentage of viable cells was calculated relative to the untreated control. Linear regression dose-response (variable slope) analysis was used to calculate the concentration at which the drugs induce 50% cell death, an IC₅₀ value, for each extract using Prism Software version 9.0. Standard deviations will be reported for IC₅₀ values representing>3 biological replicates (3 technical replicates/biological replicates) per compound.

Lactate accumulation assay. Lactate production in the medium was detected using the Lactate Assay Kit (BioVision, Mountain View, CA, USA). Specifically, an equal number of MIA PaCa-2 cells (4×10⁵-/well) were seeded in standard DMEM growth medium in a 6-well Nunclon plate and permitted to adhere overnight. The next day, cells were washed with PBS and treated with compound or vehicle control (v/v %) for six hours in a treatment media comprising DMEM with varied glucose and pyruvate concentrations and without serum and phenol red. At the end of 6 h, two μL of culture medium was taken for the lactate assay and diluted 100 folds with the lactate assay buffer. The cells were collected and lysed, and the lysate was used for protein quantification. A Biovision Lactate assay kit (K-607) was used, and the lactic acid secreted in the medium was determined per the manufacturer protocol with fluorescence measured at Ex/Em=535/587 nm. A standard curve was used to quantitate the lactic acid in the culture medium. Results were normalized based on the total protein. Experiments were performed in triplicate and repeated three times. The data were normalized to untreated cells (control), and the percent lactate production was calculated as (lactate in control wells—lactate in the experimental group)/control group×100%.

Protein extraction. After six hours of treatment with the inhibitors, cells were gently scrapped and lysed using ice-cold RIPA buffer supplemented with protease inhibitors. The lysate was incubated (4° C. for 20 minutes) and then centrifuged (15,000 g, 20 min, 4° C.) to collect the supernatant containing the total proteins. The total protein in cell lysates was then quantified using the BCA assay kit (Catalog# 23227).

Western blot analysis. Briefly, compound-treated cells were lysed in RIPA buffer supplemented with protease/phosphatase inhibitors. Equal amount of protein as assessed by BCA protein assay kit was subjected to SDS gel electrophoresis followed by western blotting.

Statistical analysis. Each experiment was run in triplicates and repeated 3 times. Statistical analysis was performed using prism 9.0 software (GraphPad). Differences between the groups were explored by Student's t test for two group comparisons or by one-way ANOVA for multiple groups, followed by Dunnett's multiple comparison test. P value<0.05 was considered significant.

Thus, in accordance with the present disclosure, there have been provided compounds, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A composition, comprising:
at least one compound, wherein the at least one compound is an inhibitor of Lactate dehydrogenase-A (LDHA), and wherein the at least one compound has a structure represented by Formula I: 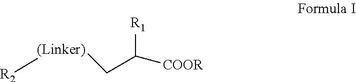
or an enantiomer, stereoisomer, or pharmaceutically acceptable salt thereof,
wherein:
R is H, an alkyl group, or a substituted phenyl group;
R₁ is selected from a phenyl group; an alkyl group; a substituted phenyl group; an aromatic ring; a heteroaromatic ring; a fused bicyclic group; a biphenyl group; or a 3-7 membered carbocyclic or alicyclic ring incorporating at least one heteroatom;
R₂ is selected from a phenyl group; a substituted phenyl group; an aromatic ring; a heteroaromatic ring; a bicyclic group; a tricyclic group; a 3-7 membered alicyclic ring incorporating at least one heteroatom; a carbocyclic ring; or a disubstituted carbocyclic ring; and
Linker comprises at least one of an amide group; a reverse amide group; an ether group; an alkyl group; or a 3-7 membered carbocyclic, alicyclic, heterocyclic, aromatic, or heteroaromatic ring.

2. The composition of claim 1, wherein the at least one compound is further defined as having a structure represented by one of Formulas IV-VII: 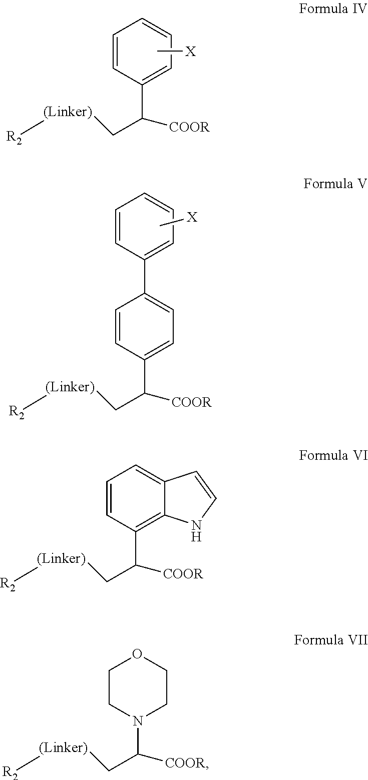
wherein X is selected from the group consisting of H and at least one ortho, meta, or para substitution.

3. The composition of claim 2, wherein the at least one substitution of X is selected from the group consisting of a halogen, an alkyl group, a cyano group, a nitro group, a trifluoromethyl group, a hydroxyl group, an amino group, a carboxylic acid group, a carbocyclic ring, an alicyclic ring incorporating at least one heteroatom, a heteroaromatic ring, a substituted heteroaromatic ring, and combinations thereof.

4. The composition of claim 1, wherein the Linker is an amide group, and wherein the at least one compound is further defined as having a structure represented by Formula VIII or XXXI:

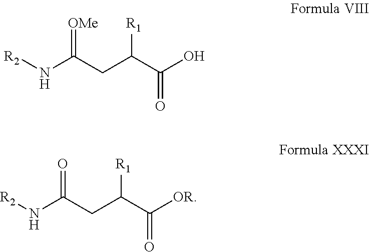

5. The composition of claim 4, wherein the at least one compound has a structure represented by Formula LXII or LXIII:

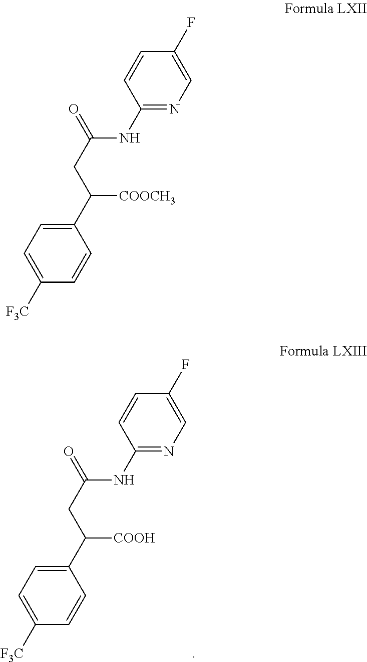 [SMILES: COC(=O)C(CC(=O)Nc1ccc(F)cn1)c1ccc(C(F)(F)F)cc1 ; O=C(CC(C(=O)O)c1ccc(C(F)(F)F)cc1)Nc1ccc(F)cn1]

6. The composition of claim 1, wherein the Linker is a heterocyclic ring, and wherein the at least one compound is further defined as having a structure represented by one of Formulas XV-XIX and XXI-XXII:

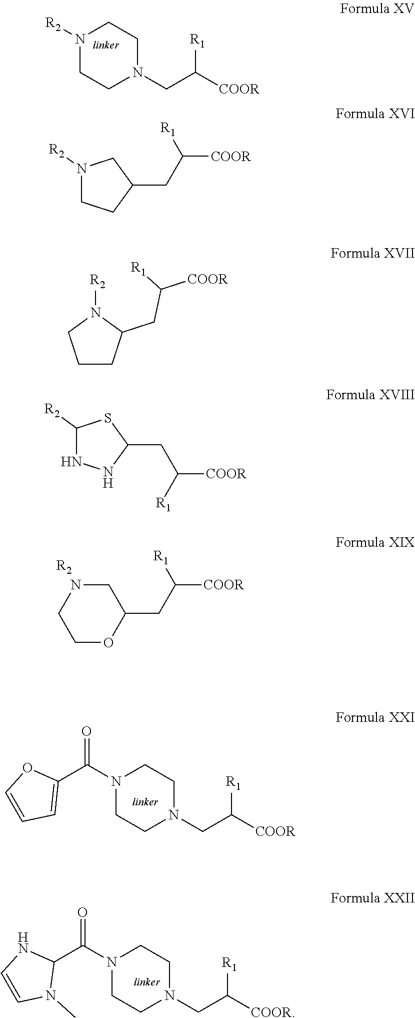

7. The composition of claim 1, wherein the Linker comprises an amide group and a ring that is connected to a carbonyl group, and wherein the at least one compound is further defined as having a structure represented by Formula XXIII: 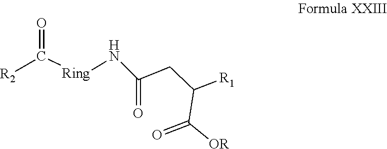
wherein the Ring comprises a 3-7 membered carbocyclic, alicyclic, heterocyclic, aromatic, or heteroaromatic ring.

8. The compound of claim 7, wherein the at least one compound has a structure represented by one of Formulas IX, XI, XIII, XLV, LIII, LIX, LX, LXXVI, LXXVIII, LXXXVIII, or LXXXIX:

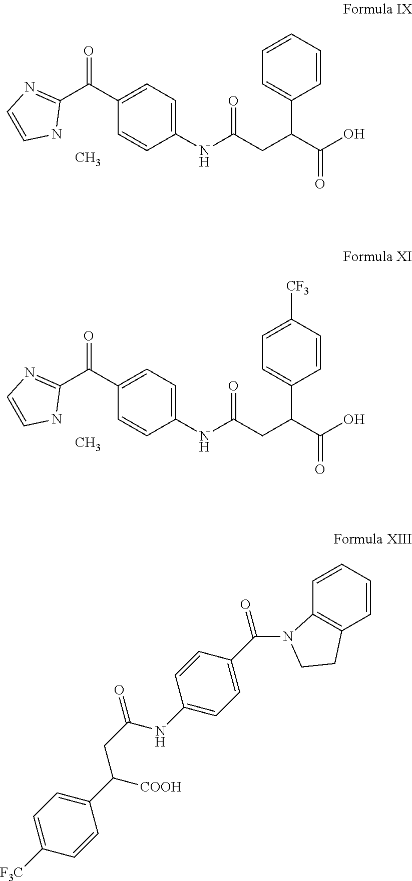

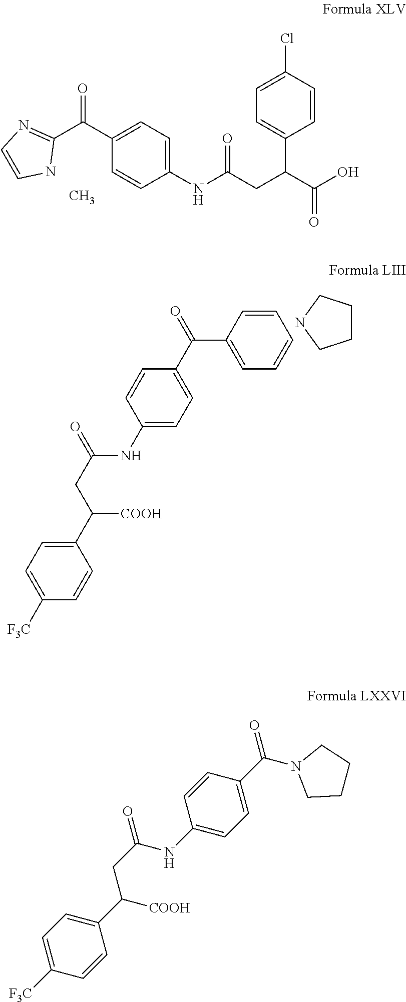

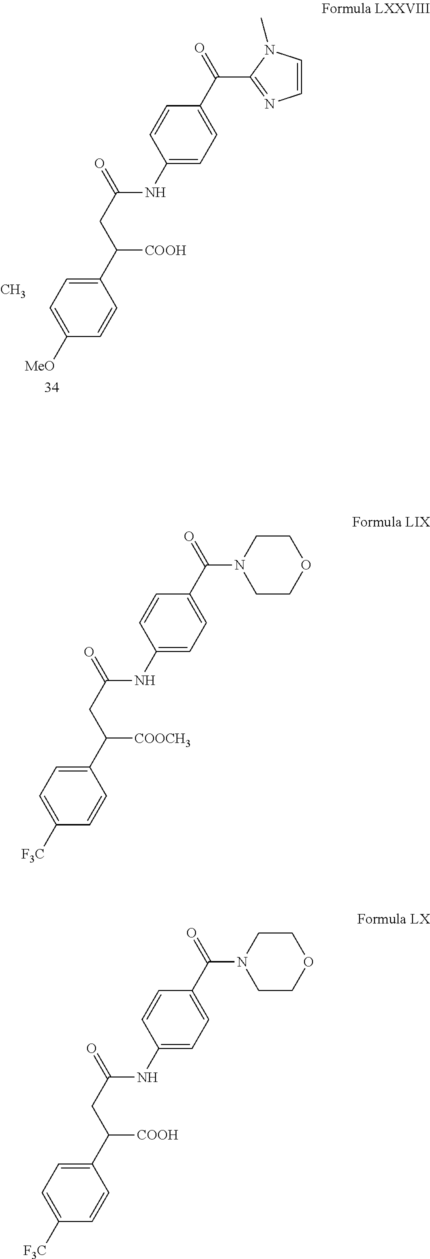

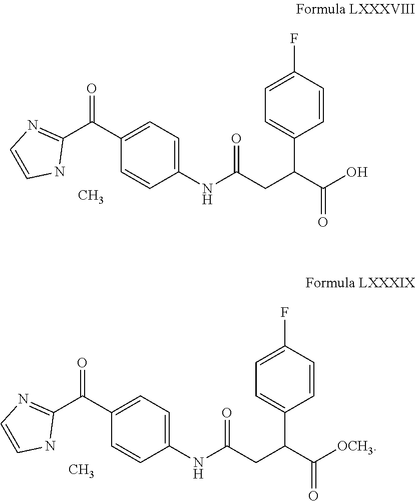

9. The composition of claim 1, wherein the Linker comprises an amide group and a ring, wherein the Ring and R₂ form a cyclized system, and wherein the at least one compound is further defined as having a structure represented by Formula XXVII: 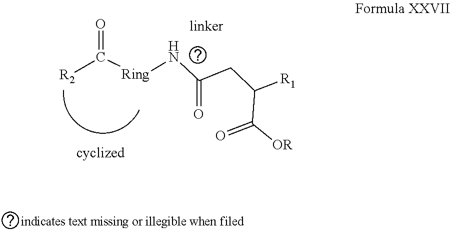
wherein the Ring comprises a 3-7 membered carbocyclic, alicyclic, heterocyclic, aromatic, or heteroaromatic ring.

10. The composition of claim 1, wherein the at least one compound has an IC₅₀ against LDHA in a range of from about 0.01 μM to about 10 μM.

11. The composition of claim 1, wherein the compound has a cellular viability IC₅₀ against pancreatic cancer cells in a range of from about 1 μM to about 30 μM.

12. A method of inhibiting LDHA expression and/or enzymatic activity in at least one cell, comprising:
exposing the at least one cell to the composition of claim 1.

13. A method of reducing viability of cancer cells, comprising:
administering the composition of claim 1 to the cancer cells.

14. The method of claim 13, wherein the cancer cells are selected from the group consisting of breast cancer cells, lung cancer cells, cerebral cancer cells, bladder cancer cells, thyroid cancer cells, prostate cancer cells, colon cancer cells, rectal cancer cells, pancreatic cancer cells, stomach cancer cells, liver cancer cells, uterine cancer cells, hepatic cancer cells, renal cancer cells, prostate cancer cells, cervical cancer cells, ovarian cancer cells, lymphoma cells, neuroblastoma cells, melanoma cells, myeloma cells, Wilm's tumor cells, leukemia cells, astrocytoma cells, glioma cells, retinoblastoma cells, and combinations thereof.

15. The method of claim 13, wherein the cancer cells are pancreatic cancer cells.

16. A pharmaceutical composition, comprising:
the composition of claim 1; and
at least one pharmaceutically acceptable carrier.

17. A method, comprising:
administering the pharmaceutical composition of claim 16 to a subject in need thereof.

18. The method of claim 17, wherein the subject has cancer.

19. The method of claim 18, wherein the cancer is selected from the group consisting of breast cancer, lung cancer, cerebral cancer, bladder cancer, thyroid cancer, prostate cancer, colon cancer, rectal cancer, pancreatic cancer, stomach cancer, liver cancer, uterine cancer, hepatic cancer, renal cancer, prostate cancer, cervical cancer, ovarian cancer, lymphoma, neuroblastoma, melanoma, myeloma, Wilm's tumor, leukemia, astrocytoma, glioma, retinoblastoma, and combinations thereof.

20. The method of claim 19, wherein the subject has pancreatic cancer.

21-34. (canceled)
